(19) United States
(12) Patent Application Publication
(10) Pub. No.: US 2003/0176455 A1
(43) Pub. Date: Sep. 18, 2003

(54) Method of inhibiting cell death

(75) Inventor: Steven J. Adelman, Doylestown, PA (US)

Correspondence Address: WYETH, PATENT LAW GROUP, FIVE GIRALDA FARMS, MADISON, NJ, 07940, US

(73) Assignee: Wyeth, Madison, NJ (US)

(21) Appl. No.: 10/384,808

(22) Filed: Mar. 10, 2003

Related U.S. Application Data
(60) Provisional application No. 60/364,188, filed on Mar. 13, 2002.

(51) Int. Cl. A61K 31/4745

(52) U.S. Cl. 514/291

(57) ABSTRACT

This invention provides a method of inhibiting cell death in a mammal in need thereof, which comprises providing said mammal with an effective amount of a rapamycin

This application claims priority from copending provisional application Serial No. 60/364,188 filed Mar. 13, 2002, the entire disclosure of which is hereby incorporated by reference.

### BACKGROUND OF THE INVENTION

This invention relates the use of a rapamycin in the inhibition of cell death, particularly following cellular regeneration.

Recent published information supports the concept that that appropriate cell types can repopulate damaged tissue, leading to their repair. Two examples of this are demonstrated by the publications of Orlic (Nature 2001;410:701-705) and Quaini (NEJM 2002;346:5-15). In the publication by Orlic et al., it was found that bone marrow cells can regenerate infarcted myocardium. In the Quaini publication, it was demonstrated that transplanted donor hearts could be populated by recipient cells possessing both stem cell markers and mature myocyte characteristics. Although this information supports the concept of tissue regeneration, in many disease processes, there is an ongoing immune or autoimmune response that will destroy the repopulated cells. Examples of this can be seen in Type I diabetes where the insulin secreting islet cells are initially destroyed by autoimmune processes, and in chronic heart failure where there is a significant loss of functional cardiac myocytes through immune destruction. In these two instances, repopulating stem cells will simply be destroyed again by the immune system.

Rapamycin is a macrocyclic triene antibiotic produced by Streptomyces hygroscopicus, which was found to have antifungal activity, particularly against Candida albicans, both in vitro and in vivo [C. Vezina et al., J. Antibiot. 28, 721 (1975); S. N. Sehgal et al., J. Antibiot. 28, 727 (1975); H. A. Baker et al., J. Antibiot. 31, 539 (1978); U.S. Pat. No. 3,929,992; and U.S. Pat. No. 3,993,749]. Additionally, rapamycin alone (U.S. Pat. No. 4,885,171) or in combination with picibanil (U.S. Pat. No. 4,401,653) has been shown to have antitumor activity.

The immunosuppressive effects of rapamycin have been disclosed in FASEB 3, 3411 (1989). Cyclosporin A and FK-506, other macrocyclic molecules, also have been shown to be effective as immunosuppressive agents, therefore useful in preventing transplant rejection [FASEB 3, 3411 (1989); FASEB 3, 5256 (1989); R. Y. Calne et al., Lancet 1183 (1978); and U.S. Pat. No. 5,100,899]. R. Martel et al. [Can. J. Physiol. Pharmacol. 55, 48 (1977)] disclosed that rapamycin is effective in the experimental allergic encephalomyelitis model, a model for multiple sclerosis; in the adjuvant arthritis model, a model for rheumatoid arthritis; and effectively inhibited the formation of IgE-like antibodies.

Rapamycin is also useful in preventing or treating systemic lupus erythematosus [U.S. Pat. No. 5,078,999], pulmonary inflammation [U.S. Pat. No. 5,080,899], insulin dependent diabetes mellitus [U.S. Pat. No. 5,321,009], skin disorders, such as psoriasis [U.S. Pat. No. 5,286,730], bowel disorders [U.S. Pat. No. 5,286,731], smooth muscle cell proliferation and intimal thickening following vascular injury [U.S. Pat. Nos. 5,288,711 and 5,516,781], adult T-cell leukemia/lymphoma [European Patent Application 525,960 A1], ocular inflammation [U.S. Pat. No. 5,387,589], malignant carcinomas [U.S. Pat. No. 5,206,018], cardiac inflammatory disease [U.S. Pat. No. 5,496,832], and anemia [U.S. Pat. No. 5,561,138].

### DESCRIPTION OF THE INVENTION

This invention provides a method of inhibiting cell death in a mammal in need thereof, which comprises providing an effective amount of a rapamycin to said mammal. In particular, this invention is useful in inhibiting cell death following cellular regeneration in response to a disease, or traumatic injury causing cell death.

As defined herein, the term “a rapamycin” defines a class of immunosuppressive compounds which contain the basic rapamycin nucleus (shown below). The rapamycins of this invention include compounds which may be chemically or biologically modified as derivatives of the rapamycin nucleus, while still retaining immunosuppressive properties. Accordingly, the term “a rapamycin” includes esters, ethers, oximes, hydrazones, and hydroxylamines of rapamycin, as well as rapamycins in which functional groups on the rapamycin nucleus have been modified, for example through reduction or oxidation. The term “a rapamycin” also includes pharmaceutically acceptable salts of rapamycins, which are capable of forming such salts, either by virtue of containing an acidic or basic moiety.

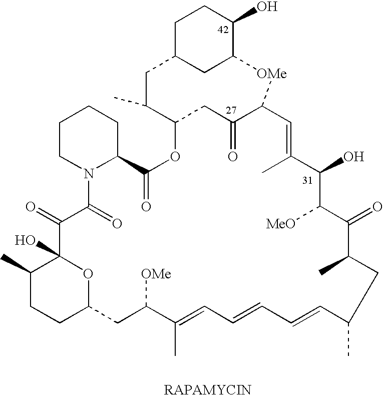

It is preferred that the esters and ethers of rapamycin are of the hydroxyl groups at the 42- and/or 31-positions of the rapamycin nucleus, esters and ethers of a hydroxyl group at the 27-position (following chemical reduction of the 27-ketone), and that the oximes, hydrazones, and hydroxylamines are of a ketone at the 42-position (following oxidation of the 42-hydroxyl group) and of 27-ketone of the rapamycin nucleus.

Preferred 42- and/or 31-esters and ethers of rapamycin are disclosed in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above.

Preferred 27-esters and ethers of rapamycin are disclosed in U.S. Pat. No. 5,256,790, which is hereby incorporated by reference. The preparation of these esters and ethers are disclosed in the patents listed above.

Preferred oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines are disclosed in the above listed patents. The preparation of 42-oxorapamycin is disclosed in U.S. Pat. No. 5,023,263, which is hereby incorporated by reference.

Particularly preferred rapamycins include rapamycin [U.S. Pat. No. 3,929,992], rapamycin 42-ester with 3-hydroxy-2-(hydroxymethyl)-2-methylpropionic acid [U.S. Pat. No. 5,362,718], and 42-O-(2-hydroxy)ethyl rapamycin [U.S. Pat. No. 5,665,772].

When applicable, pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable aids when the rapamycin contains a suitable basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when the rapamycin contains a suitable acidic moiety.

As used in accordance with this invention, the term “providing,” with respect to providing a compound or substance covered by this invention, means either directly administering such a compound or substance, or administering a prodrug, derivative, or analog which will form the equivalent amount of the compound or substance within the body.

As used in accordance with this invention, the term “cellular regeneration” means new cell growth to repopulate cells or tissue that have been damaged or killed as a result of a disease or traumatic injury.

In accordance with this inventions, rapamycins are useful in inhibition of cell death, particularly following cellular regeneration after cell death or traumatic injury. In particular, the rapamycins of this invention are useful in inhibiting cell death of regenerating cells in damaged cells or tissue that has resulted from autoimmune diseases, diseases of aging, traumatic injury. Diseases or traumas such as coronary artery disease, congestive heart failure, diabetes (Types I and II), Alzheimer's disease, dimentias, memory loss, rheumatoid arthritis, neuropathy, cartlidge disorders, and spinal injury or degeneration all cause localized cell death. Such cells include, but are not limited to, pancreatic beta cells, vascular tissue, cardiac tissue, brain cells, including neurons, hepatic cells, liver cells, skin cells, bone cells and spinal tissue. Either upon treatment with various drugs, or by the body's natural regenerative processes the dead or damaged tissue can regenerate. Absent an effective treatment to inhibit cell death, the disease process will kill the regenerating cells. The rapamycins of this invention are useful in inhibiting cell death of the regenerating pancreatic beta cells, vascular tissue, cardiac tissue, brain cells, including neurons, hepatic cells, liver cells, skin cells, bone cells or spinal tissue.

It is understood that the effective dosage of the rapamycin may vary depending upon the particular compound utilized, the mode of administration, the condition, and severity thereof, of the condition being treated, as well as the various physical factors related to the individual being treated. As used in accordance with invention, satisfactory results may be obtained when the rapamycin is administered in a daily oral dosage of from about 5 μg to 0.75 mg per kilogram of body weight. The projected daily dosages are expected to vary with route of administration.

When a rapamycin is used as part of a combination regimen, dosages of each of the components of the combination are administered during a desired treatment period. The components of the combination may administered at the same time; either as a unitary dosage form containing both components, or as separate dosage units; the components of the combination can also be administered at different times during during a treatment period, or one may be administered as a pretreatment for the other.

Such doses may be administered in any manner useful in directing the active compounds herein to the recipient's bloodstream, including orally, via implants, parenterally (including intravenous, intraperitoneal and subcutaneous injections), rectally, intranasally, vaginally, and transdermally. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. It is more preferred that poloxamer 188 is used as the surface modifying agent. Oral formulations herein may utilize standard delay or time release formulations to alter the absorption of the active compound(s). Preferred oral formulations of rapamycins are disclosed in U.S. Pat. Nos. 5,559,121; 5,536,729; 5,989,591; and 5,985,325, which are hereby incorporated by reference.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils. Preferred parenteral formulations for administering a rapamycin are disclosed in U.S. Pat. Nos. 5,530,006; 5,516,770; and 5,616,588, which are hereby incorporated by reference.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

What is claimed is:

1. A method of inhibiting cell death following cellular regeneration in a mammal in need thereof, which comprises providing said mammal with an effective amount of a rapamycin.

2. The method according to claim 1, wherein the cellular regeneration occurs following cell or tissue damage caused by coronary artery disease, congestive heart failure, diabetes (Types I and II), Alzheimer's disease, dimentias, memory loss, rheumatoid arthritis, neuropathy, cartlidge disorders, or spinal injury or degeneration.

3. The method according to claim 2, wherein the rapamycin is rapamycin.

4. The method according to claim 2, wherein the rapamycin is a ester, ether, oxime, hydrazone, or hydroxylamine of rapamycin

5. The method according to claim 4, wherein the rapamycin is a 42-ester or 42-ether of rapamycin.

6. The method according to claim 5, wherein the rapamycin is rapamycin 42-ester with 3-hydroxy-2-(hydroxymethyl)-2-methylpropionic acid.

7. The method according to claim 5, wherein the rapamycin is 42-O-(2-hydroxy) ethyl rapamycin.

8. A method of inhibiting cell death of pancreatic beta cells, vascular tissue, cardiac tissue, brain cells, including neurons, hepatic cells, liver cells, skin cells, bone cells or spinal tissue following cellular regeneration of such cells or tissue in a mammal in need thereof, which comprises providing said mammal an effective amount of a rapamycin.

9. The method according to claim 8, wherein the rapamycin is rapamycin.

10. The method according to claim 8, wherein the rapamycin is a ester, ether, oxime, hydrazone, or hydroxylamine of rapamycin

11. The method according to claim 10, wherein the rapamycin is a 42-ester or 42-ether of rapamycin.

12. The method according to claim 11, wherein the rapamycin is rapamycin 42-ester with 3-hydroxy-2-(hydroxymethyl)-2-methylpropionic acid.

13. The method according to claim 11, wherein the rapamycin is 42-O-(2-hydroxy) ethyl rapamycin.

14. A method of enhancing cell regeneration in a mammal in need thereof, which comprises providing said mammal an effective amount of a rapamycin.

15. The method according to claim 14, wherein the rapamycin is rapamycin.

16. The method according to claim 14, wherein the rapamycin is a ester, ether, oxime, hydrazone, or hydroxylamine of rapamycin

17. The method according to claim 16, wherein the rapamycin is a 42-ester or 42-ether of rapamycin.

18. The method according to claim 117, wherein the rapamycin is rapamycin 42-ester with 3-hydroxy-2-(hydroxymethyl)-2-methylpropionic acid.

19. The method according to claim 17, wherein the rapamycin is 42-O-(2-hydroxy) ethyl rapamycin.
